(19) United States
(12) Patent Application Publication
(10) Pub. No.: US 2019/0011464 A1
(43) Pub. Date: Jan. 10, 2019

(54) METHOD FOR THE IN-OVO SEX IDENTIFICATION OF CHICKS

(71) Applicant: OVONDO e. K., Leipzig (DE)

(72) Inventor: Almuth EINSPANIER, Leipzig (DE)

(21) Appl. No.: 16/064,434

(22) PCT Filed: Dec. 22, 2016
(86) PCT No.: PCT/EP2016/082483

§ 371 Date: Jun. 20, 2018

(30) Foreign Application Priority Data
Dec. 22, 2015 (DE) 10 2015 226 490.4

(51) Int. Cl. G01N 33/74; G01N 33/08; G01N 21/78

(52) U.S. Cl. CPC G01N 33/743; G01N 21/78; G01N 33/08

(57) ABSTRACT

The present invention relates to a method for the in-ovo sex identification of chicks, comprising the following steps: extracting a sample solution of allantoic fluid from an egg; uniting the sample solution extracted from the egg with an oestrone-derivative-specific antibody and a conjugate solution with a conjugate binding to the antibody; and identifying the oestrone derivative concentration of the extracted allantoic fluid by means of the conjugate bound to the antibody. In order to accelerate the identification of the oestrone derivative concentration the oestrone-derivative-specific antibody is surface-bound.

The present invention relates to a method for the in-ovo sex identification of chicks.

A generic method is for example known from the dissertation by Dr. Anne Weissmann, University of Leipzig, from 2014 titled “in-ovo sex identification in laying hybrids by endocrine analysis of the allantoic fluid”.

At present, around 40 million male day-old chicks are killed every year in Germany, as only the production of laying hens is in the foreground with the laying hybrids and, therefore, the male chicks have no further use.

The dissertation by Anne Weissmann described above suggests using the concentration of oestrone sulfate in the embryonic urine to identify the corresponding sex of the developing chicks in the respective egg.

For this, without damaging or destroying the egg, approximately 50 μl of allantoic fluid is extracted from the egg between the 7^{th} and 10^{th} incubation day using a 1 ml syringe (insulin syringe: Omnican 40; B. Braun, Melsungen, Germany) and the concentration of oestrone sulfate contained in the allantoic fluid is identified.

On the basis of the oestrone sulfate concentration thus identified, the sex of the chick in the egg can then be identified and the egg, if appropriate, to the extent that the oestrone sulfate concentration indicates a male chick, can be disposed of before the chick's feeling of pain that occurs on about the 11^{th} day.

In the dissertation, it has been shown that from the 9^{th} incubation day and especially also on the 10^{th} incubation day, significantly different oestrone sulfate concentrations develop in the eggs, if a female or male embryonic chick is contained in them.

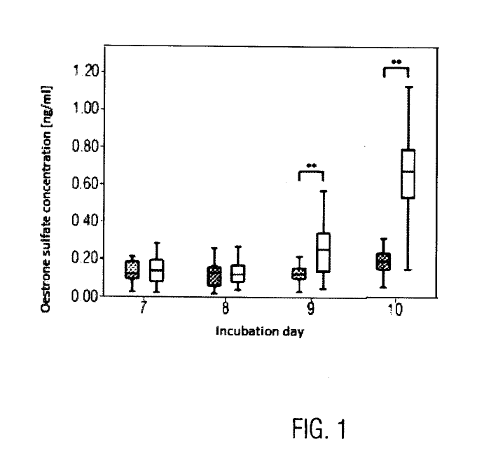

This is shown in FIG. 1 which is taken from the previously described dissertation.

In FIG. 1, the corresponding bars in the diagrams show the measured concentrations of oestrone sulfate in eggs with female embryos (light grey bars) and the concentrations of oestrone sulfate in eggs with male embryos (dark grey bars).

Thus, on the 9^{th} day, the average concentration of oestrone sulfate is between 0.125 (median value) for male embryos and 0.169 ng/ml for female embryos.

On the 10^{th} incubation day, the average concentration of oestrone sulfate in male embryos is between 0.193 (median value) and 0.667 ng/ml in female embryos. The corresponding error tolerances are also marked in FIG. 1.

The method described in the dissertation is explained below

The detection of oestrone sulfate concentration is based on a so-called enzyme immunoassay (ELISA) by the double antibody technique using 96 well plates.

The microtiter plates (96 well plates) are coated with 100 μl oestrone sulfate non-specific antibody (goat-anti-rabbit IgG). This fluid undergoes an overnight incubation and the microtiter plate subsequently is washed.

Afterwards, the actual analysis of the medium (allantois) takes place.

Said ELISA is based on the double antibody technique; an enzyme-labelled oestrone sulfate antibody complex (reaction product is oestrone sulfate from the medium to be identified (e.g. allantois) to which the oestrone sulfate-specific antibody and then the enzyme bind) reacts with an oestrone sulfate-unspecific antibody bound to a surface (e.g. titer plate substrate).

The oestrone sulfate-unspecific antibody is a goat-anti-rabbit IgG.

The allantoic fluid extracted is diluted with a buffer and mixed with an enzyme solution and a solution with an oestrone sulfate-specific antibody (E1S).

As oestrone sulfate-specific antibody, anti-oestrone glucuronide is used.

The enzyme used is oestrone glucuronide peroxidase which binds to the oestrone sulfate-specific antibody. The oestrone sulfate in the medium to be identified (e.g. allantois) also binds to the oestrone sulfate-specific antibody to which the enzyme is bound.

After the reaction on the surface (the microtiter plates), it is rinsed. This ensures that only the (enzyme-labelled) antibody-bound oestrone sulfate from sample and standard solution remains, because it is bound to the surface.

The enzyme used is able to activate a color reaction. The intensity of the dyeing can then be used to identify the concentration of oestrone sulfate in the extracted allantoic fluid by extinction measurement in the photometer.

The intensity of the dyeing is in relation to known concentrations (=standard curve).

The points of the standard curve are between 0.0125 and 5.0 ng/ml.

The enzyme solution which is combined with the sample (e.g. allantois) containing the oestrone sulfate and the oestrone sulfate-specific antibody (E1S) has a concentration of 1:100,000.

During the 3-hour reaction time, the reaction product (enzyme-labelled oestrone sulfate antibody complex) is bound to the surface-bound hormone-unspecific antibody.

The method described above for the identification of oestrone sulfate in the allantoic fluid functions by means of the double antibody technique and the final results as to whether the embryo is male or female are available after 4 hours.

The solution of the oestrone sulfate-specific antibody (E1S) which is combined with the sample containing the oestrone sulfate and the enzyme solution has a concentration of 1:125,000.

The method described above is quite complex and takes quite a long time until the correspondingly high oestrone sulfate concentrations in the allantoic fluid extracted can finally be identified.

As a result, the method is not routinely applicable for large-scale industrial use, where high throughput and rapid analysis of a large number of eggs are required.

Accordingly, it is the objective of the present invention to improve the aforementioned method so that it can also be applied on an industrial scale.

For this purpose, the invention proposes a method with the features of claim 1.

This method further develops the above described method known from the dissertation to the extent that the oestrone-derivative-specific antibody is surface bound. According to this, a so-called direct coating with the hormone-specific antibody is available. Thus, the so-called double antibody technique described above for the state of the art cannot necessarily be used. Since the oestrone-derivative-specific antibody does not react with the oestrone derivative in solution first, but is supplied already bound to a surface, the process for identifying the concentration of oestrone derivatives can be significantly accelerated. Remarkably, tests have shown that it is possible to bind the oestrone-derivative-specific antibody to a surface. In this way, the oestrone derivative concentration can be generally identified within a maximum of 2 hours.

The identification of the oestrone derivative concentration is generally described below. An example of a specific oestrone derivative is oestrone sulfate. The identification of the oestrone sulfate concentration is described in particular as an example for the identification of the concentration of a specific oestrone derivative. Any oestrone degradation product or oestrone itself can be used as an oestrone derivative. On the basis of the identification of the oestrone derivative concentration, a distinction can be made between male and female embryos. An oestrone derivative-specific antibody is sufficient for binding the oestrone derivative. Insofar as it is referred to the identification of the oestrone sulfate concentration as an example, an oestrone-specific antibody can be used as antibody.

In addition to standard (unmodified) specific antibodies, specific antibodies are also known as specific antibody conjugates. Said antibody conjugates have a specific modification compared to the standard (unmodified) antibodies, via which e.g. another group (a conjugate) can be bound and/or conjugated. However, these specific antibody conjugates are still specific to the corresponding oestrone derivative. Accordingly, the term is understood functionally and does not rule out further modification. Monoclonal or polyclonal antibodies can be used as antibodies. Polyclonal antibodies can be produced in different animal species (rabbit, goat or sheep) against the corresponding oestrone sulfate or its derivatives. The hybridoma technique in rabbits or rodents is used to produce monoclonal antibodies.

In the present case, a conjugate is understood as a group that binds to the (modified or unmodified) antibody in addition to the specific derivative to which the antibody is specific. Said binding can be bound e.g. via bridges with glucuronide, other linkers are also possible. Depending on whether or not an oestrone derivative is additionally bound to this antibody, this conjugate can have a different color, catalyze a color reaction differently, and/or form a complex with different color. This can be used, for example, to identify the concentration of the oestrone derivative. The dyeing is only to be understood as an example. It is sufficient that the concentration of the oestrone derivative can be identified with the aid of the conjugate. This can also be done by means of a video analysis via which a change in the state is detected. Depending on the oestrone derivative bound to the antibody, the conjugate or the entire complex of conjugate, antibody and oestrone derivative may have different activity. This allows the corresponding concentration of oestrone derivative to be identified.

According to a preferred embodiment according to claim 2, the oestrone-derivative-specific antibody may be present directly bound to a surface. The oestrone-derivative-specific antibody is bound directly to a surface. Thus, in contrast to the state of the art, no unspecific antibody is provided between the surface and the specific antibody.

According to a preferred embodiment according to claim 3, the conjugate may contain a colloidal marker. Said colloidal marker can be colloidal gold, colloidal latex or another colloidal substance. This colloidal marker can form the conjugate itself or be only a part of the conjugate. If gold is used as colloidal marker, it is called an immunogold conjugation. Due to the intensive dye of the colloidal gold on the one hand and the specific binding properties of the antibodies on the other hand, antibody-gold conjugates can be used well for the identification of the oestrone derivative concentration. Gold conjugates (the antibody to which colloidal gold is conjugated directly or indirectly) exhibit good stability, as the binding between protein and gold particles is relatively strong. Thus, a higher sensitivity is achieved. Latex conjugates containing latex colloids or being a latex colloid can also be used, as the label can be practically tailored. Latex particles can be produced in any color.

According to a preferred embodiment according to claim 3, the conjugate may be selected from the following group of compounds: gold-labelled steroid-protein compound, colloidal gold, latex-labelled steroid-protein compound, colloidal latex. Steroid-protein compounds are e.g. enzymes or modified enzymes. The enzymes are e.g. chemically modified so that the colloidal marker can bind better to them. However, any other modification is also conceivable. Examples of steroid-protein compounds are: Oestrone glucuronide peroxidase, oestrone carboxymethyl oxime, oestrone carboxymethyl ether. This conjugate can then be bound to the antibody. These conjugates are available in solution, for example. In this context, colloidal mixtures are also referred to as solutions.

According to a preferred embodiment according to claim 4, the surface to be coated with the oestrone-derivative-specific antibody may be a glass, fiberglass, paper or polypropylene surface.

According to a preferred embodiment according to claim 5, the oestrone-derivative-specific antibody may be an oestrone sulfate-specific antibody to identify the oestrone sulfate concentration of extracted allantoic fluid. As previously described, this variant is a subgroup of the oestrone-derivative-specific antibody. As it is referred to the oestrone sulfate-specific antibody below, this generally also applies to the oestrone-derivative-specific antibody (in modified or unmodified form for conjugation with the conjugate) and/or not only to oestrone sulfate but to any oestrone derivatives.

According to a preferred embodiment according to claim 6, the conjugate solution may be an enzyme solution containing an enzyme capable of activating an added dye and that the identification of the oestrone sulfate concentration of the allantoic fluid extracted is carried out by photometrically identifying the concentration of the dye activated by the enzyme.

As described above, this variant in which an enzyme is used as conjugate is a subgroup of the general form of the conjugate. A colloidal marker may also be bound to the enzyme. Insofar as reference is made below to “enzyme” in relation to the dependent claims, any other conjugate may also be used. The concentrations and/or advantages also apply in the general form to the conjugates used.

The oestrone sulfate-specific antibody is also surface bound. According to this, a so-called direct coating with the hormone-specific antibody is available. Therefore, it is not necessary to use the so-called double antibody technique described above for the state of the art. Due to the fact that the oestrone sulfate-specific antibody does not react with the oestrone sulfate in solution first, but is already bound to a surface, the process for identifying the concentration of oestrone sulfate can be significantly accelerated. Remarkably, tests have shown that it is possible to bind the oestrone sulfate-specific antibody to a surface. In this way, the oestrone sulfate concentration can be identified within a maximum of 2 hours.

In the following, with respect to dependent claims 8 to 20, the variant in which the oestrone derivative is oestrone sulfate and accordingly, the antibody is an oestrone sulfate-specific antibody is described; wherein an enzyme which is capable of activating an added dye is used as conjugate and, that the identification of the oestrone sulfate concentration of the extracted allantoic fluid is carried out by photometrically identifying the concentration of the dye activated by the enzyme.

According to a preferred embodiment according to claim 8, the allantoic fluid sample solution and the enzyme solution, which has a dilution of between 1:60,000 and 1:90,000, can be added to the surface-bound oestrone sulfate-specific antibody.

Due to the lower dilution (higher concentration) of the enzyme solution in direct coating compared to the aforementioned state of the art (double antibody technique), it has been shown that when the sample solution (allantoic fluid) and the enzyme solution are brought into contact with the surface coated with the oestrone sulfate-specific antibody, a faster and thus more effective reaction between enzyme or oestrone sulfate from the sample and the hormone-specific antibody takes place.

Anti-oestrone glucuronide is preferably used as an oestrone sulfate-specific antibody. For immunization (antibody production), a carrier molecule BSA (bovine albumin) is preferably coupled to the oestrone by means of glucuronide. Accordingly, the oestrone sulfate-specific antibody is preferably produced against oestrone glucuronide.

Oestrone glucuronide peroxidase is preferably used as the enzyme. Preferred ranges of dilution of the enzyme solution can be between 1:70,000 and 1:80,000, in particular at approx. 1:75,000+1-1,000. Insofar as it is referred to a dilution of the enzyme solution, this dilution can be carried out with the aid of water (preferably demineralized) or a buffer solution.

According to a preferred embodiment according to claim 9, 2 μl to 50 μl allantoic fluid can be extracted from the egg. Another preferred quantity range is 5 μl to 20 μl.

Surprisingly, it has been shown that even with a lower extraction quantity compared to the state of the art described in claim 3, the different oestrone sulfate concentrations can still be identified reliably.

The less allantoic fluid (e.g. <50 μl) is extracted, the less or no deterioration in the hatching rate could be detected compared to eggs that were not sampled.

According to a preferred embodiment according to claim 10, the allantoic fluid extracted from the egg may be combined undiluted with the oestrone sulfate-specific antibody and the enzyme.

The samples (allantois) are therefore preferably used undiluted in the direct coating method. Thus the allantoic fluid, essentially extracted as it is from the egg, can be used. Essentially as it is does not exclude mechanical filtering. Preferably, however, such filtering is also omitted, as it has remarkably turned out that even an unfiltered and undiluted sample does not adversely affect the subsequent reaction.

This undiluted allantoic fluid can then be applied to the coated surface together with the enzyme.

According to a preferred embodiment according to claim 11, between 2 μl and 50 μl sample solution and between 30 μl and 70 μl enzyme solution can be combined. Other preferred values are between 5 μl and 20 μl, in particular 50 μl. Preferably, equal amounts of sample solution and enzyme solution are mixed and brought into contact with the coated surface.

According to a preferred embodiment according to claim 12, a solution of the oestrone sulfate-specific antibody may be applied to the surface to be coated in a dilution of 1:90,000 to 1:150,000.

Preferred ranges of dilution of the oestrone sulfate-specific antibody may be between 1:100,000 and 1:130,000, in particular approx. 1:125,000+/−1,000.

In order to be able to immobilize the oestrone sulfate-specific antibody on the surface, the concentration should be significantly higher than if the antibody solution is used for an in-situ reaction only.

According to a preferred embodiment according to claim 13, to coat the surface with the oestrone sulfate-specific antibody, the solution of the oestrone sulfate-specific antibody may act on the surface to be coated for 4 to 14 hours. It has been shown that the best results of immobilization can be achieved, if the solution of the oestrone sulfate-specific antibody acts on the surface for 8 to 13 hours.

Following this coating, a subsequent coating with 0.1% BSA solution (bovine serum albumin) can preferably be carried out. This can be done as follows, in particular, if the surface to be coated is a microtiter plate: 280 μl+/−50 μl BSA solution (fraction V, company Serva) is incubated per cavity for approx. 30 minutes, then emptied.

According to a preferred embodiment according to claim 14, the coated surface can only be washed once with polysorbate 80 solution after acting of the solution of the oestrone sulfate-specific antibody and, if necessary, the BSA. If the surface to be coated is the cavity of a microtiter plate, a single rinse with approx. 300 μl per cavity of the microtiter plate is sufficient. A rinse can be carried out with a quantity of 50 μl to 300 μl.

Polysorbate 80 is a polyoxyethylated compound derived from sorbitol and oleic acid. Polysorbate 80 is also known as TWEEN 80.

It has been shown that after a single washing, essentially all non-immobilized oestrone sulfate-specific antibodies could be removed and that enough oestrone sulfate-specific antibodies were also present in immobilized form and were not washed out.

According to a preferred embodiment according to claim 15, the surface to be coated may be a cavity of a micro-test plate or a membrane.

Said micro-test plates are well known and have a variety of cavities (also called wells) in which the sample solution and the enzyme solution can be mixed.

These cavities serve as micro-reactors for the identification. Preferably, 96-well, 384-well micro-test plates (company nunc) can be used.

Alternatively, instead of using such cavities, a membrane can also be used, which can then be installed in a test cassette.

Said procedure is a modification of the technique known as the lateral-flow technique.

According to a preferred embodiment according to claim 16, the sample solution and the enzyme solution can be added to the coated cavity of the micro-test plate and the reaction mixture poured out after the reaction.

Preferably, the cavities coated with oestrone sulfate-specific antibodies are directly mixed with the corresponding amounts of sample solution and enzyme solution.

It has been shown that the micro-test plates with the coated cavities can be stored for months even at below −17° C., in particular at approx. −17 to 23° C., without changing unfavorably with regard to the subsequent reaction with the oestrone sulfate.

Thus, it is possible for the first time to prepare the micro-test plates with the coated cavities long before the identification.

According to a preferred embodiment according to claim 17, the generated immobilized reaction product can only be washed twice with polysorbate 80 after reaction between oestrone sulfate-specific antibody, oestrone sulfate in the sample and enzyme.

It has been shown that two washes are sufficient to remove all undesired molecules not bound to the oestrone sulfate-specific antibody so that the bound oestrone sulfate can be identified very precisely with regard to its concentration. If the surface to be coated is the cavity of a microtiter plate, rinsing twice with 300 μl, in particular 300 μl per cavity of the microtiter plate, is sufficient.

According to a preferred embodiment according to claim 18, the washed immobilized reaction product can be combined with a solution containing a dye that can be activated by the enzyme and the dye concentration can be photometrically identified after a predetermined time.

Said dye which can be activated by the bound enzyme, can be produced as follows. A substance A of H20rUrea/NarHP04/Citric acid is mixed together with a substance B of tetramethylbenzidine/DMSO/citric acid, each in equal parts, and applied to a coated surface, in particular the cavity. The enzyme catalyzes a color reaction through which the enzyme concentration and thus indirectly the oestrone sulfate concentration can be concluded.

By means of photometric methods, the relative dye concentration can be determined with reference to the standard points (0.125-1.0 ng/ml).

Standard points (reference solutions) with known concentrations of oestrone sulfate (0.125-1.0 ng/ml) are prepared which react with the enzyme and the immobilized oestrone sulfate-specific antibody in the same way as the extracted allantoic fluid. A calibration curve is created from the measured photometric data (absorption). The photometric data (absorption) measured on the sample to be examined are compared with the calibration curve to identify the concentration of the oestrone sulfate.

The photometric identification is preferably carried out as follows:

The enzyme (e.g. oestrone glucuronide peroxidase) from the enzyme solution binds to the surface-bound antibody.

After the color reaction, the antibody-bound enzyme produces the highest extinction measurement value in the photometer, if no oestrone sulfate (from the calibration series or allantois sample) is bound to the antibody.

With increasing oestrone sulfate concentration, less enzyme binds to the surface-bound antibody, whereby the extinction decreases with the later color reaction.

From this, preferably by computer evaluation, the concentration of oestrone sulfate can be identified.

Thus, the dyeing (measured extinction) is inversely proportional to the calibration curve.

According to preferred embodiment according to claim 19, the photometric measurement of the color reaction can be performed to identify the dye concentration in the cavity of the micro-test plate.

This can further accelerate the method.

According to a preferred embodiment according to claim 20, the micro-test plate may contain a plurality of cavities, and the allantoic fluid extracted from different eggs may be placed in different cavities, and the oestrone sulfate concentration in the different eggs may be identified automatically by automating the photometric measurement in the different cavities and/or automating the step of combining enzyme, sample solution into the coated cavity.

This can further accelerate the method.

Dependent claims 21 to 33 describe the general variant of the method which has been further explained in relation to claims 1 to 6. The corresponding concentrations and the procedure can be slightly different in comparison to the special variant. This variant is not limited to a photometric identification but any method can be used with which the concentration of the oestrone derivative can be concluded with the help of the conjugate. A method in which a video camera is used to identify the concentration-dependent properties is also conceivable.

Further preferred embodiments of the invention results from the examples discussed below in connection with the drawing.

Therein:

FIG. 1 shows the measured concentrations of oestrone sulfate in eggs with female embryos as well as the concentrations of oestrone sulfate in eggs with male embryos known from the state of the art,

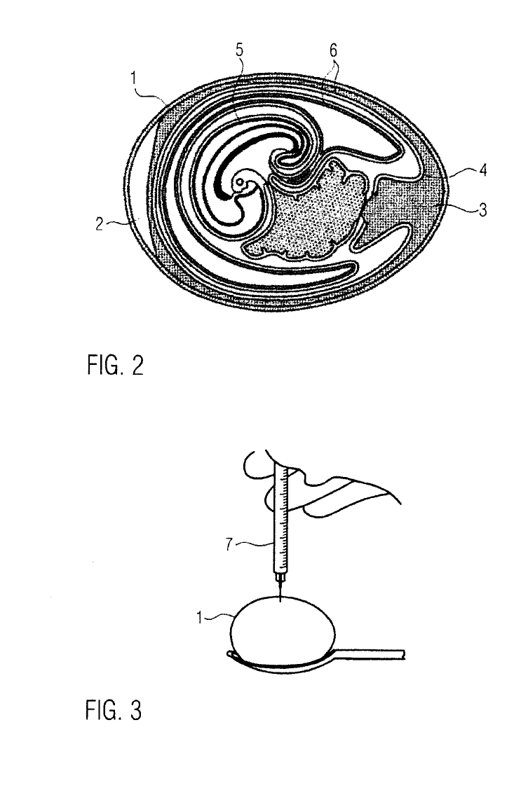

FIG. 2 shows a schematic illustration of a chicken embryonic membrane on the 10^{th} to 11^{th} day of incubation,

FIG. 3 shows the extraction of allantoic fluid by means of an insulin syringe, and

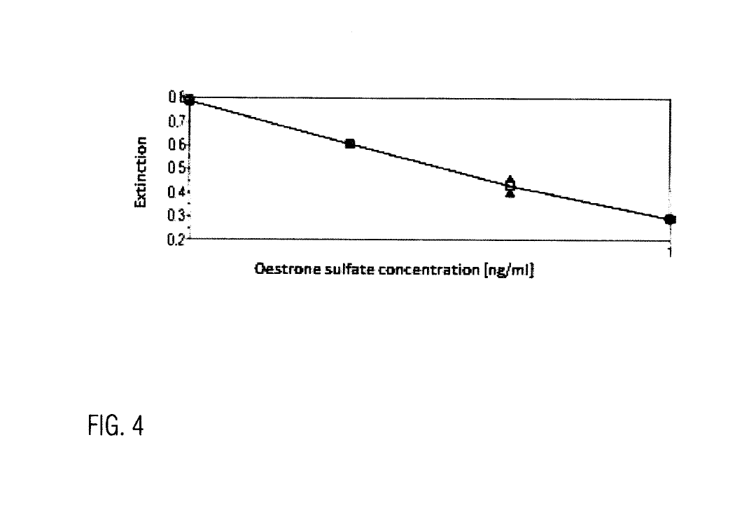

FIG. 4 shows a calibration curve for the identification of the oestrone sulfate content according to the present invention.

FIG. 2 shows an embryonic coat of a chicken on the 10^{th} to 11^{th} incubation day.

The egg shown schematically in its cross-sectional view has a calcareous shell 1 with a shell skin, within the calcareous shell 1 an air chamber 2 and protein 3 are enclosed.

The so-called chorion 4 in which the embryonic chick 5 and the allantois 6 are housed, is stored in the protein.

The metabolic products of the embryo are mainly excreted in the form of ammonium urea and uric acid. Another degradation product is the oestrone sulfate described above. Even if the concentration of oestrone sulfate is identified by the state of the art, any oestrone degradation product or oestrone itself can be used to make a distinction between male and female embryos. Therefore, only one oestrone-derivative-specific antibody is defined in claim 1. Therefore, the inventive method is also generally described for oestrone derivatives.

In the following, only the variant in which the oestrone derivative is oestrone sulfate and accordingly, the antibody is an oestrone sulfate-specific antibody, is described as a special example; wherein an enzyme which is capable of activating an added dye is used as conjugate and that the identification of the oestrone sulfate concentration of the allantoic fluid extracted is carried out by photometrically identifying the concentration of the dye activated by the enzyme.

However, this description also applies to any oestrone derivative or conjugate and any method can be used to identify the concentration of oestrone derivative. A photometric measurement is not essential.

FIG. 3 shows an egg into which an insulin syringe 7 has been injected in such a position that appropriate fluid can be withdrawn from the allantois.

Examples of individual components are described below.

### Preparation of the Sample Solution

Allantoic fluid samples of 2 μl to 50 μl, preferably 5 μl to 20 μl, are extracted from the egg by means of a tip.

The Allantoic Fluid Extracted is Used Undiluted and Thus Forms the Sample Solution, which is applied to the coated surface with the enzyme solution.

### Preparation of the Enzyme Solution

The enzyme used here is oestrone glucuronide peroxidase.

The oestrone sulfate in the sample binds to the oestrone sulfate-specific antibody to which the enzyme also binds.

Preferred areas of dilution of the enzyme solution can be between 1:60,000 and 1:90,000, preferably between 1:70,000 and 1:80,000, in particular at approx. 1:75,000+/−1,000.

The enzyme was diluted with buffer.

The buffer solution contained 7.12 g sodium hydrogen phosphate (Merck, Germany), 8.5 g sodium chloride (Merck, Germany), 1.0 g bovine serum albumin (Serva, Germany) and 1000 ml water. The buffer solution had a pH value of 7.2.

### Surface Coating/Storage

A solution of the oestrone sulfate-specific antibody can be applied to the surface to be coated at a dilution of 1:90,000 to 1:150,000, preferably between 1:100,000 and 1:130,000, in particular at approx. 1:125,000+/−1,000.

Anti-oestrone glucorunide, i.e. the antibody is directed against oestrone glucuronide (BSA) and was obtained in rabbits, can preferably be used as an oestrone sulfate-specific antibody.

The surface to be coated was a cavity of a micro-test plate. Preferably, 96-well, 384-well, micro-test plates (company nunc) can be used.

As an alternative to the micro-test plates, a membrane to which the oestrone sulfate-specific antibody is bound can also be used, and the solutions can be placed on the membrane in cassettes. This is a modification of the technique known as lateral flow assay, which is a competitive assay system. The surface to be coated can be glass, fiberglass, paper or polypropylene.

In this case, the solutions are applied to glass, fiberglass, paper, polypropylene or membrane surfaces to start the reaction. This system does not require a rinsing process as described below. After the marker solution has been added, a color change takes place, which is documented.

Between 50 μl and 150 μl, in particular 100 μl solution of the oestrone sulfate-specific antibody, were placed in each cavity and left overnight at room temperature.

Afterwards the cavities of the micro-test plates were emptied with between 100 μl and 500 μl, in particular 300 μl assay buffers for 30 minutes at 150/min.

The assay buffer was then emptied from the microtiter plate.

The cavities of the plates are now coated with the oestrone sulfate-specific antibody.

These plates can be stored without damage at at least −17° C., in particular at approx. −17 to −23° C. for months before they are used.

### Reaction

The sample solution and the enzyme solution were placed in the coated cavities and incubated for 75 minutes while oscillating. After this time, everything had reacted stoichiometrically and the oestrone sulfate was bound to the oestrone sulfate-specific antibody on the surface.

Equal amounts of between 2 μl and 50 μl sample solution and between 30 μl and 70 μl enzyme solution were added together. Other preferred values are between 5 μl and 15 μl, in particular 50 μl.

After the reaction, the micro-test plate was emptied and washed twice with TWEEN 80 solution

### Method for Identifying the Concentration of Oestrone Sulfate

The washed immobilized reaction product was combined with a solution containing a dye that can be activated by the enzyme and the concentration of the dye was determined photometrically after a predetermined time.

Said dye which can be activated by the bound enzyme can be produced as follows.

A substance A of H20rurea/NarHP04/citric acid is mixed with a substance B of tetramethylbenzidine/DMSO/citric acid in equal parts and placed on the rinsed surface. The enzyme catalyzes a color reaction through which the hormone concentration and thus indirectly the oestrone sulfate concentration can be concluded.

The relative dye concentration can be determined by photometric methods.

Standard solutions with known concentrations of oestrone sulfate are prepared which react with the enzyme and the immobilized oestrone sulfate-specific antibody in the same way as the extracted allantoic fluid. A calibration curve is created from the measured photometric data (absorption). The photometric data (absorption) measured on the sample to be examined are compared with the standard curve in order to identify the concentration of the oestrone sulfate.

To identify the oestrone sulfate concentration, calibration samples of known quantities of oestrone sulfate were prepared and treated in the same way as described above. The calibration curve shown in FIG. 4 was obtained from the data measured for these calibration robes.

By comparing the results of the photometric measurement of samples with unknown oestrone sulfate content with the calibration curve, the oestrone sulfate concentration in the sample can be concluded.

The spectrometer used was a so-called Viktor 21420 multi-label counter, the measured values were determined with the “Workout” software (Perkin Elmer).

### Oestrone Sulfate Concentration for Sex Identification

With the method described above, differences in the oestrone sulfate concentration of the allantoic fluid of 0.1 ng/ml can be distinguished which is not possible with the previously known method.

This means that a more precise identification can be carried out between female and male embryos.

For clarification, this is shown in tables 1 and 2 below. Thereby table 1 shows the concentrations of oestrone sulfate or oestradiol in the allantoic fluid samples extracted at different incubation times.

TABLE 1
Concentrations of oestrone sulfate or oestradiol in allantoic
fluid samples extracted at different incubation times.

| Sampling day/ | | Oestrone sulfate (ng/ml) | | | | Oestradiol (pg/ml) | | | |
|---|---|---|---|---|---|---|---|---|---|
| Embryonic day | Sex | median | P 25 | P75 | p | median | P25 | P75 | p |
| 7 | male | 0.11 | 0.07 | 0.14 | 0.91 | — | — | — | — |
| | female | 0.12 | 0.53 | 0.14 | | — | — | — | |
| 8 | male | 0.12 | 0.10 | 0.19 | 0.29 | 40.95 | 28.38 | 50.75 | 0.56 |
| | female | 0.15 | 0.11 | 0.23 | | 32.40 | 29.95 | 38.90 | |
| 9 | male | 0.14 | 0.09 | 0.17 | <0.01 | 157.65 | 54.35 | 326.75 | 0.01 |
| | female | 0.21 | 0.14 | 0.35 | | 329.50 | 215.18 | 370.25 | |
| 10 | male | 0.17 | 0.14 | 0.22 | <0.01 | 101.75 | 43.68 | 116.10 | <0.01 |
| | female | 0.68 | 0.57 | 0.81 | | 158.90 | 125.85 | 214.85 | |

In table 1 above median, P25 and P75 show the respective quantiles between P25 and P75 as 50% of the values of the measured distribution.

For example, the median value of oestrone sulfate concentration in male embryos on the 7^{th} incubation day is 0.11 ng oestrone sulfate per ml sample extracted. The median value of oestrone sulfate concentration in female embryos on the 7^{th} incubation day is 0.12 ng oestrone sulfate per ml sample extracted.

For example, the median value of oestrone sulfate concentration in male embryos on the 9^{th} incubation day is 0.14 ng oestrone sulfate per ml sample extracted. The median value of oestrone sulfate concentration in female embryos on the 8^{th} incubation day is 0.21 ng oestrone sulfate per ml sample extracted.

Corresponding concentrations are also given for oestradiol in the table.

Table 2 shows the limit values of oestrone sulfate concentrations indicating a male embryo or a female embryo.

TABLE 2
Limit values of oestrone sulfate concentrations
indicating a male embryo or a female embryo.

| Sampling day/ | | Oestrone sulfate (ng/ml) |
|---|---|---|
| Embryonic day | Sex | GW (limit values) |
| 7 | male | No difference |
| | female | |
| 8 | male | ≤0.10 |
| | female | >0.10 |
| 9 | male | ≤0.17 |
| | female | >0.17 |
| 10 | male | ≤0.26 |
| | female | >0.26 |

Table 2 above indicates the differences in the oestrone sulfate concentration depending on the incubation days.

On the 7^{th} incubation day, no difference can be determined with the method according to the invention.

On the 8^{th} incubation day, the limit for male embryos is 0, 10 ng/ml or less oestrone sulfate in the allantoic fluid. A concentration of more than 0.10 ng/ml oestrone sulfate is a female embryo.

Using the method according to the invention, a difference in the oestrone sulfate concentration can be detected even earlier and the identification can also be carried out more rapidly.

### REFERENCE SIGN LIST

Calcareous shell 1
Air chamber 2
Protein 3
Chorion 4
Embryonic chick 5
Allantois 6
Insulin syringe 7

1. Method for the in-ovo sex identification of chicks, comprising the steps:
extracting a sample solution of allantoic fluid from an egg;
uniting the sample solution extracted from the egg with an oestrone-derivative-specific antibody and a conjugate solution with a conjugate binding to the antibody; and
identifying the oestrone derivative concentration of the extracted allantoic fluid by means of the conjugate bound to the antibody,
wherein the oestrone-derivative-specific antibody is present directly bound to a surface, wherein the surface to be coated with the oestrone-derivative-specific antibody may be a membrane, glass, fiberglass, paper or polypropylene surface.

2-33. (canceled)

34. Method according to claim 1, wherein:
the conjugate contains a colloidal marker, and/or the conjugate is selected from the following group of compounds: gold-labelled steroid-protein compound, colloidal gold, latex-labelled steroid-protein compound, colloidal latex.

35. Method according to claim 1, wherein:
the oestrone-derivative-specific antibody is an oestrone sulfate-specific antibody and the oestrone sulfate concentration is identified from the extracted allantoic fluid.

36. Method according to claim 35, wherein:
the conjugate solution is an enzyme solution containing an enzyme capable of activating an added dye, and that the identification of the oestrone sulfate concentration of the allantoic fluid extracted is carried out by photometrically identifying the concentration of the dye activated by the enzyme.

37. Method according to claim 36, wherein:
the sample solution and the enzyme solution, which has a dilution of between 1:60,000 and 1:90,000, can be added to the surface-bound oestrone sulfate-specific antibody, and/or
that 2 μl to 50 μl sample solution are extracted from the egg, and/or
that the sample solution is combined undiluted with the oestrone sulfate-specific antibody and the enzyme solution, and/or
that between 2 μl and 50 μl sample solution and between 30 μl and 70 μl enzyme solution are combined.

38. Method according to claim 36, wherein:
a solution of the oestrone sulfate-specific antibody may be applied to the surface to be coated in a dilution of 1:90,000 to 1:150,000, and/or
that for coating the surface with the oestrone sulfate-specific antibody, the solution of the oestrone sulfate-specific antibody may act on the surface to be coated for 4 to 14 hours, and/or
that the coated surface is washed only once with polysorbate 80 solution after exposure of the solution of the oestrone sulfate-specific antibody.

39. Method according to claim 36, wherein:
the sample solution and the enzyme solution is added to the coated cavity of the micro-test plate and the reaction mixture is poured out after the reaction, and/or
that the generated immobilized reaction product is washed only twice with polysorbate 80 after reaction between oestrone sulfate-specific antibody, oestrone sulfate and enzyme, and/or
that the washed immobilized reaction product is combined with a solution containing a dye that can be activated by the enzyme and the dye concentration is photometrically identified after a predetermined time, and/or
that the photometric measurement of the color reaction is performed to identify the dye concentration in the cavity of the micro-test plate.

40. Method according to at least one of claim 36, wherein:
the micro-test plate contains a plurality of cavities, and the sample solution extracted from different eggs is placed in different cavities, and the oestrone sulfate concentration in the different eggs is identified automatically by automating the photometric measurement in the different cavities and/or automating the step of combining enzyme, sample solution into the coated cavity.

41. Method according to claim 1, wherein:
the sample solution and the conjugate solution having a dilution of between 1:20,000 and 1:100,000 are added to the surface-bound oestrone-derivative-specific antibody, and/or
that 2 μl to 50 μl sample solution are extracted from the egg, and/or
that the sample solution is combined undiluted with the oestrone sulfate-specific antibody and the enzyme, and/or
that between 2 μl to 50 μl sample solution and between 30 μl and 70 μl conjugate solution are combined.

42. Method according to claim 1, wherein:
a solution of the oestrone sulfate-specific antibody is applied to the surface to be coated in a dilution of 1:90,000 to 1:150,000, and/or
that for coating the surface with oestrone-derivative-specific antibody, the solution of the oestrone-derivative-specific antibody acts on the surface to be coated for 4 to 14 hours, and/or
that after acting of the solution of the oestrone-derivative-specific antibody, the coated surface is washed only once with polysorbate 80 solution.

43. Method according to claim 36, wherein:
the sample solution and the enzyme solution are applied into the cavity of the micro-test plate and the reaction mixture is poured out after the reaction.

44. Method according to claim 35, wherein:
the generated immobilized reaction product is washed only twice with polysorbate 80 after reaction between oestrone sulfate-specific antibody, oestrone sulfate, and enzyme,

45. Method according to claim 1, wherein:
a photometric measurement to identify the concentration of oestrone derivate is carried out after a predetermined time.

46. Method according to claim 45, wherein:
the photometric measurement of the color reaction is carried out for identifying the dye concentration in the cavity of the micro-test plate.

47. Method according to claim 35, wherein:
the micro-test plate contains a plurality of cavities, and the sample solution extracted from different eggs is placed in different cavities, and the oestrone sulfate concentration in the different eggs is identified automatically by automating the photometric measurement in the different cavities and/or automating the step of combining conjugate, sample solution into the coated cavity.
